(19) United States
(12) Patent Application Publication
(10) Pub. No.: US 2003/0229007 A1
(43) Pub. Date: Dec. 11, 2003

(54) Form of human renin and its use as a target in treatments for cardiac ischemia and arrhythmia

(76) Inventors: Roberto Levi, New York, NY (US); Randi B. Silver, New York, NY (US)

Correspondence Address: HOFFMANN & BARON, LLP, 6900 JERICHO TURNPIKE, SYOSSET, NY, 11791, US

(21) Appl. No.: 10/449,848

(22) Filed: May 30, 2003

Related U.S. Application Data
(60) Provisional application No. 60/385,116, filed on May 30, 2002.

(51) Int. Cl. A61K 39/395; A61K 38/17

(52) U.S. Cl. 514/2; 424/146.1; 514/381

(57) ABSTRACT

The present invention provides a method for treating a human suffering from myocardial ischemia, cardiac arrhythmia, or both. The method comprises administering locally to a heart of a human an effective amount of an enzyme inhibitor that inhibits formation of angiotensin II in the heart. The invention also provides an isolated human renin of about 32-36 kDa.

This application asserts priority to U.S. Provisional Application No. 60/385,116 filed May 30, 2002. The specification of U.S. Provisional Application No. 60/385,116 is hereby incorporated by reference in its entirety.

### BACKGROUND OF THE INVENTION

Angiotensin (Ang) is a peptide that plays an important role in many processes including regulating blood volume, blood pressure, plasma volume, sympathetic neural activity, and thirst response. The biologically active form of Ang, Ang II, exerts its effects by binding Ang II receptors (e.g., AT₁ and AT₂). Ang II is produced by the conversion of Ang I which is formed as a result of the action of renin on angiotensinogen.

The conversion of angiotensinogen to Ang II is referred to as the renin-angiotensin system (RAS). Traditionally, RAS has been viewed as a circulating system. In circulating RAS, liver-derived angiotensinogen is cleaved by kidney-derived renin to form Ang I. Ang I is then converted to Ang II by angiotensin-converting enzyme (ACE). The conversion of Ang I by ACE typically occurs on the luminal side of the vascular endothelium.

In addition to circulating RAS, many tissues, including the heart, are capable of producing Ang II locally. Thus, Ang II may mediate various autocrine, paracrine and intracrine effects in the heart.

Renin, angiotensinogen, ACE, and Ang II receptors are present in the myocardium (Dostal et al., Circ. Res. 1999, 85:643-650; Bader et al., J. Mol. Med. 2001, 79:76-102). In the heart, Ang II is also generated by non-ACE enzymes, including chymase (Ihara et al., Cardiology 2000, 94:247-253). It is not known whether the generation of angiotensin in the heart occurs as a result of the action of circulating renin or locally produced renin.

There is, in fact, no convincing evidence for renin synthesis in the heart. Thus, the existence of local RAS in the heart is controversial (Danser et al., Cardiovascular Res. 1999, 44:252-265; Danser et al., J. Mol. Cell. Cardiol. 2002, 34:1463-1472).

It has been reported that Ang II is a potent facilitator of norepinephrine (NE) release from peripheral (Zimmerman, Circ. Res. 1962, 11:780-787) and cardiac sympathetic nerve endings (Seyedi et al., Circ. Res. 1997, 81:774-784). Some controversy remains on this subject, since according to recent literature, Ang II is not a local mediator of cardiac sympathetic activity in the in vivo porcine heart (Lameris et al., Hypertension 2002, 40:491-497). Local Ang II formation increases during myocardial ischemia (Jalowy et al., J. Am. Soc. Nephrol. 1999, 10 (Suppl 11):S129-136). However, the mechanism of release of NE from cardiac sympathetic nerve endings by Ang II is not known. Moreover, it is not known where in the heart Ang II is generated.

Myocardial ischemia is a deficiency of oxygenated blood to the cells of the heart. The deficiency of blood may, for example, be caused by functional constriction or obstruction of a blood vessel. The lack of oxygen and/or reduced availability of nutrient substrates and inadequate removal of metabolites may result in tissue damage, for example, apoptosis and/or necrosis of cells.

Myocardial ischemia is often caused by a reduction in coronary blood flow relative to myocardial demand. The reduction in blood flow may result from a variety of reasons, and typically occurs as a result of atherosclerosis. Ischemia of the heart can lead to cardiac arrhythmia.

Cardiac arrhythmia is a change in the regular beat of a heart. For example, the heart may skip a beat, beat faster, or beat slower. There are many different types of arrhythmia which are usually defined by where the arrhythmia occurs in the heart.

Arrhythmia typically originates in the atria or the ventricles. Examples of arrhythmias which originate in the atria include sinus arrhythmia, sinus tachycardia, sick sinus syndrome, premature supraventricular contractions or premature atrial contractions, supraventricular tachycardia, paroxysmal atrial tachycardia, atrial flutter, atrial fibrillation, and Wolff-Parkinson-White syndrome. Examples of arrhythmias which originate in the ventricles include premature ventricular complexes, ventricular tachycardia, and ventricular fibrillation.

In chronic hypertension, circulating renin has been reported to be produced in excess by the kidneys. Circulating plasma renin has a molecular weight of about 38-42 kDa.

In hypertension, renin has long been targeted for therapeutic intervention. Use of medications that target the renin-angiotensin system for the treatment of hypertension can often achieve the desired decrease in blood pressure. Thus, oral administration of ACE inhibitors and Ang II receptor blockers are used in the treatment of hypertension to indirectly block the action of renin.

As stated above, local Ang II formation increases in myocardial ischemia. Thus, it would also be desirable to directly or indirectly block the action of renin in myocardial ischemia and cardiac arrhythmia. However, myocardial ischemia and cardiac arrhythmias are generally refractory to these anti-hypertensive therapies.

Thus, there is a need for new methods of treating patients suffering from myocardial ischemia, cardiac arrhythmia, or both.

### SUMMARY OF THE INVENTION

These and other objectives have been met by the present invention, by providing a method for treating a human suffering from myocardial ischemia, cardiac arrhythmia, or both. The method comprising administering locally to a heart of the human an effective amount of an enzyme inhibitor that inhibits formation of angiotensin II in the heart.

In another embodiment, the invention provides an isolated human renin of about 32-36 kDa.

In yet a further embodiment, the invention provides a method for discovering drugs for treating myocardial ischemia or cardiac arrhythmia in humans. The method comprises providing a compound selected from a plurality of compounds for testing; and determining whether the compound specifically inhibits renin.

In yet another embodiment, the invention provides a method for screening drug candidates for treating myocardial ischemia or cardiac arrhythmia in humans. The method comprises providing a compound from a plurality of compounds for testing; and determining whether the compound specifically inhibits chymase.

### BRIEF DESCRIPTION OF THE FIGURES

FIG. 1. Purity of cardiac sympathetic nerve endings. Western blot of human right atrial homogenate and synaptosomal preparation, run in parallel, with an antibody directed against sarcomeric myosin heavy chains, MF20 (Bader et al. J. Cell. Biol. 1982, 95:763-770). Equal amounts of protein (1.5 μg) for both atrial homogenate and synaptosomes were run in parallel and in triplicate on the same gel. Bands correspond to a molecular weight of ˜205 kDa. Only a minimal presence of myosin was found in the synaptosomal preparation (O.D.: 768±281 and 9,744±705 for synaptosomes and atrial homogenate, respectively).

FIG. 2. NE release from human right atrium incubated in ischemic conditions in the absence or presence of renin inhibitors. Specimens were incubated either without any drug or with increasing concentrations of pepstatin-A (panel A) and BILA 2157BS (panel B). Points (mean±S.E.M.) represent the total NE released during 70 min of ischemia. NE release after 70 min of ischemia in the absence of renin inhibitors (=100%) was 3.5±0.26 pmol/mg protein (n=12). *P<0.05 and **P<0.01, significantly different from ischemia control by one-way ANOVA followed by Dunnett multiple comparison test.

FIG. 3. NE release from synaptosomes isolated from surgical specimens of human right atrium. Synaptosomes were incubated for 70 min either in normoxic (normal HBS, gassed with 95% O₂ and 5% CO₂) or ischemic conditions (glucose-free HBS, containing 3 mM sodium dithionite and gassed with 95% N₂ and 5% CO₂), in the absence or presence of desipramine (DMI, 300 nM), the Na⁺/H⁺ exchanger inhibitor EIPA (30 μM), renin inhibitors pepstatin-A (PEP, 30 μM) and BILA 2157BS (BILA, 100 nM), the ACE inhibitor enalaprilat (ENAL, 3 μM) or the Ang II AT₁-receptor antagonist EXP 3174 (EXP, 300 nM). Bars (mean±S.E.M.; n=8-36) represent total NE released during each 70-min incubation period. *p<0.05 vs control normoxic NE release and †p<0.05 vs control ischemic NE release, respectively, by one-way ANOVA followed by Dunnett multiple comparison test.

FIG. 4. NE release from synaptosomes isolated from surgical specimens of human right atrium. Synaptosomes were incubated for 70 min either in normoxic (normal HBS, gassed with 95% O₂ and 5% CO₂) or ischemic conditions (glucose-free HBS, containing 3 mM sodium dithionite and gassed with 95% N₂ and 5% CO₂), in the absence or presence of angiotensinogen (A, 400 nM) alone or with the renin inhibitor BILA 2157BS (BILA, 100 nM), the ACE inhibitor enalaprilat (ENAL, 3 μM) or the Ang II AT₁-receptor antagonist EXP 3174 (EXP, 300 nM). Bars (mean±S.E.M.; n=8-28) represent total NE released during each 70-min incubation period. *p<0.05 vs control ischemic NE release in the absence of angiotensinogen, and †p<0.05 vs ischemic NE release in the presence of angiotensinogen, by one-way ANOVA followed by Dunnett multiple comparison test.

FIG. 5. Detection of renin in sympathetic nerve endings isolated from human right atrium. Western blot of normoxic vs. ischemic synaptosome preparations with an antibody directed against renin, BR1-5 (Campbell et al. Hypertension 1996, 27:1121-1133). Equal amounts of protein (1.0 μg) for both normoxic and ischemic synaptosomes were run in parallel and in triplicate on the same gel. Bands correspond to a molecular weight of approximately 34 kDa. (O.D.: 820±135 and 2,159±339 for normoxic and ischemic synaptosomes, respectively). Thus, renin activity increased ˜three-fold after 70-min ischemia.

### DETAILED DESCRIPTION OF THE INVENTION

The invention is based on the unexpected discovery of a local RAS in sympathetic nerve endings (SNE) in the heart. The inventors have discovered that Ang I is generated by locally produced renin, and then converted to Ang II in cardiac SNE. The locally produced renin is different from circulating renin.

The invention relates to a method for treating a human suffering from myocardial ischemia, cardiac arrhythmia, or both. The types of myocardial ischemia and cardiac arrhythmia in accordance with the present invention have been discussed in the background section.

The method comprises administering locally to a heart of a human an effective amount of an enzyme inhibitor that inhibits formation of angiotensin II in the heart. The enzyme target can be any enzyme involved in the formation of Ang II in the heart. An inhibitor may, for example, function by reducing the concentration or activity of its target enzyme.

The inhibitor can indirectly inhibit Ang II formation by, for example, inhibiting Ang I formation. Alternatively, the inhibitor can directly inhibit Ang II formation. An example of an enzyme which is involved in the formation of Ang I in the heart is renin. An example of an enzyme which is involved in the formation of Ang II in the heart is chymase.

The renin inhibitor can be any compound which inhibits renin. Renin inhibitors are well known and include, for example: pepstatin A, BILA 2157 BS the structure of which is described in Duan et al. Can J Physiol Pharmacol 73:1512-1518 (1995), ciprokiren Ro44-9375 the structure of which is described in Fischli et al. Hypertension 24(2) 163-169 (1994), A74273 the structure of which is described in Lin et al. Am Heart J 131:1024-1034 (1996), remikiren Ro42-5892 (ibid.), enalkiren 64662 (ibid.), CGP-38560 (ibid.), CGP-29287 the structure of which is described in Frischman et al. J Clin Pharmacol. 34:873-880 (1994), A72517 (ibid.), Abbott-72517, Aliskiren, CI-992, EMD58265, U 71038, Aliskiren—by Speedel;. CI-992—Parke Davis, Warner Lambert; EMD 58265—(5-((4-amino-1-piperidylcarbonyl)-Phe-His-ACHPA-Ile)-aminomethyl-4-amino-2-methyl-pyrimidine) from E Merck, Darmstadt, Germany; and U 71038 (L-histidinamide, 1-[(1,1 dimethylethoxy)carbonyl]-L-prolyl-L-phenylalanyl-N-[2-hydroxy-5-methyl-1-(2methylpropyl)-4-[[[2-methyl-1-[[(2-pyridinylmethyl)amino] carbonyl]butyl]amino] carbonyl]hexyl]-Nα-methyl-,[1 S-[1 R*,2 R*, 2 R*,4 R* (1 R*,2 R*)]]) supplied by Pharmacia-Upjohn (Kalamazoo, Mich., USA). The structure of these compounds are hereby incorporated by reference. Another example of a renin inhibitor is a renin antibody.

Examples of chymase inhibitors include the following: α₁-anti-trypsin, chymostatin, BBI (Bowman-Birk inhibitor), Suc-Val-Pro-Phe(p)(OPh)(2), SQN-5, MNEI, NK3201, BCEAB (4-[1-[[bis-(4-methylphenyl)-methyl]-carbamoyl]-3-(2-ethoxybenzyl)-4-oxo-azetidine-2-yloyl]-benzoic acid, methyllinderone. Another example of a chymase inhibitor is a chymase antibody.

ACE is an enzyme involved in the conversion of Ang I to Ang II. ACE inhibitors are often administered as antihypertensive agents. However, these inhibitors are not totally effective in treating cardiac ischemia and arrhythmia in humans, since the conversion of Ang I to Ang II in the heart is by mechanisms largely independent of ACE. Accordingly, the inhibitors of the invention do not include ACE.

In another embodiment, enzyme inhibitor can be a combination of enzyme inhibitors that inhibits formation of Ang I and Ang II. For example, a renin inhibitor can be administered in combination with a chymase inhibitor. Thus, the formation of both Ang I and Ang II are inhibited.

The inventors have also discovered that the locally produced Ang II acts at the sympathetic nerve endings in the heart to stimulate the activity of the sodium-hydrogen ion exchanger (NHE). Stimulation of the NHE results in an increase in intracellular sodium, thereby triggering release of norepinephrine (NE) via the NE transporter in the SNEs. Under ischemic conditions, stimulation of the NHE leads to excessive release of NE via the NE transporter (NET) acting in the reverse of the normal mode. The excessive release of NE thereby exacerbates ischemia and/or arrhythmia.

There are different types of receptors on SNE that mediate neuronal NHE activity. For example, one type of receptor can mediate enhancement of neuronal NHE activity. Therefore, the methods described above optionally further comprise administering locally to the heart an inhibitor of receptor-mediated enhancement of neuronal NHE activity. Any inhibitor of receptor-mediated enhancement of neuronal NHE activity, such as, for instance, an AT₁ angiotensin II receptor (AT₁) antagonist may be used. Examples of AT₁ receptor antagonists incude losartan (available from Merck under brand name Cozaar), irbesartan (available from Sanofi under brand name Avapro), candesartan cilexetil (available from Astra Merck under brand name Atacand), valsartan (available from Novartis under brand name Diovan), EXP3174 (the active metabolite of losartan produced in the liver), or an anti-AT₁ receptor antibody. Other angiotensin II receptor blockers are disclosed in U.S. Pat. No. 6,348,481 B1 to Inada et al. These angiotensin II receptor blockers are hereby incorporated by reference.

Alternatively, another type of receptor can mediate attenuation of neuronal NHE activity. Therefore, the methods described above optionally further comprise administering locally to the heart an agonist of a receptor which mediates attenuation of neuronal NHE activity. The attenuation of the NHE activity complements the renin and/or chymase inhibitor therapy by further attenuating the NHE activity by an independent mechanism. Any receptor which mediates attenuation of neuronal NHE activity, such as, for instance, a histamine H₃ receptor or an adenosine A₁ receptor may be the target of the agonist.

Examples of suitable histamine H₃ receptor agonists include: R-(α)-methylhistamine, imetit, immepip, SKF 91606 or Sch 50971.

Adenosine A₁ receptor agonists include, for example, adenosine analogues substituted in the 2- and N6-positions, including three classes of N6-substituents: norbornen-2-yl (series 1), norborn-2-yl (series 2) and 5,6-epoxynorborn-2-yl (series 3). Adenosine analogues substituted with fluoro, bromo, and iodo substituents are also active. See for example Hutchinson et al. Bioorg Med Chem 2002 Apr;10(4):1115-22. Adenosine analogues N(6)-cyclopentyladenosine (CPA); N(6)-cyclohexyladenosine (CHA) and N(6)-phenylisopropyladenosine (R-PIA) are also active as adenosine A₁ receptor agonists (Homayoun et al. Eur J Pharmacol 2001 Nov 2; 430(2-3):289-94).

In another embodiment, the methods described above optionally further comprise administering locally to the heart an inhibitor of NE release from the sympathetic nerve endings (SNEs). The inhibitor of NE release may be any inhibitor that inhibits NE release from SNEs, such as for example, Bretylium (a membrane stabilizer).

### Renin

It has further been found that the renin produced locally in the heart has a molecular weight of approximately 32-36 kDa. Therefore, the heart-produced renin is different from circulating renin, which has a molecular weight of about 38-42 kDa. Although different from circulating renin, heart-produced renin is bound by at least some antibodies to circulating renin. Furthermore, the heart-produced renin is inhibited by at least some inhibitors of circulating renin, such as for example, pepstatin A and BILA 2157 BS.

Accordingly, in another embodiment, the invention provides an isolated renin with an apparent molecular weight of about 32-36 kDa when run on SDS polyacrylamide gels. The renin of about 32-36 kDa is found in, and may be isolated from, cardiac synaptosomes. The cardiac synaptosomes may be isolated from sympathetic nerve endings of patients suffering from myocardial ischemia or from myocardial ischemia-induced arrhythmia.

The renin is isolated, which means that it is essentially free of other proteins. Essentially free from other proteins means that it is at least 90%, preferably at least 95% and, more preferably, at least 98% free of other proteins.

Preferably, the isolated renin is essentially pure, which means that the renin is free not only of other proteins, but also of other materials used in the isolation and identification of the protein, such as, for example, sodium dodecyl sulfate and other detergents as well as nitrocellulose paper. The essentially pure renin is at least 90% free, preferably at least 95% free and, more preferably, at least 98% free of such materials.

The isolated renin can be used, for example, in assays for screening drug candidates for treating myocardial ischemia or arrhythmia.

### Discovering Drugs

In another embodiment, the invention provides a method for discovering drugs (e.g., screening drug candidates) for treating myocardial ischemia or arrhythmia. The method comprises providing a compound (e.g., test compound) selected from a plurality of compounds for testing. The plurality of compounds is typically from a library of compounds.

The compound can be a biological molecule or a small molecule. A biological molecule is any molecule which contains a nucleic acid or amino acid sequence and has a molecular weight greater than 450. Biological molecules include nucleotides, polypeptides, peptides, and proteins.

Small molecules include organic compounds, which generally have molecular weights of approximately 450 or less. Small molecules can further include molecules that would otherwise be considered biological molecules, except their molecular weight is not greater than 450. Thus, small molecules may be lipids, oligosaccharides, oligopeptides, oligonucleotides, and their derivates, having a molecular weight of 450 or less.

It is emphasized that a small molecule can have any molecular weight. They are merely called small molecules because they typically have molecular weights less than 450.

The test compound is contacted with renin or chymase by any method known to those in the art. For example, the test compound can be incubated with renin or chymase. The renin may, for example, be circulating renin or heart-produced renin.

The next step in the method is to determine whether the test compound specifically inhibits renin or chymase. Determination of inhibition can be performed by any method known in the art. For example, assays for measuring the ability of renin to convert angiotensinogen to Ang I (see below) and assays for measuring the ability of chymase to convert Ang I to Ang II can be employed. Test compounds that inhibit renin or chymase are drug candidates.

These candidate drugs (e.g., compounds which specifically inhibit renin or chymase) can be further tested for their effectiveness in treating myocardial ischemia or arrhythmia by methods known to those in the art. For example, the further testing can be those that are routinely performed by clinicians and physicians during pre-clinical and clinical trials.

### Administration

Due to the unexpected discovery by the inventors of a local RAS in the adrenergic nerves of the heart, it is important to administer inhibitors which inhibit local RAS as close as possible to the site of NE release. Accordingly, an effective amount of the inhibitors useful in the methods of the present invention is administered locally to the heart of a human in need thereof.

The inhibitor (including a combination of inhibitors) is administered in an amount effective in treating myocardial ischemia, arrhythmia, or both by reducing the release of NE from SNE. The effective amount may, for example, be determined during pre-clinical trials and clinical trials by methods familiar to physicians and clinicians.

For the purposes of the present specification, “local delivery” of an inhibitor to the heart includes any delivery method that introduces the inhibitor directly into the heart. For example, the inhibitors of the invention may be administered by intrapericardial delivery, percutaneous intrapericardial delivery, medicated stents, or balloon catheter.

These methods of local delivery of compounds to the heart are well known to those in the art. For example, intrapericardial delivery of contrast agents for radiology and antineoplastic agents for therapy is disclosed in Maisch et al. Clin. Cardiol. 22 (Suppl. I), I-17 to I-22 (1999); Waxman et al. Cathet. Cardiovasc. Intervent 49:472-477 (2000); and in Farrell et al. Am. J. Physiol. Heart Circ. Physiol. 281:H813-H822 (2001), the methods of which are hereby incorporated by reference. Percutaneous intrapericardial drug delivery has been used for therapeutic angiogenesis (Laham et al. Clin. Cardiol. 22:(Suppl. I), I-6 to I-9 (19999) in this case for bFGF delivery), the method of which is hereby incorporated by reference.

Local delivery also includes medicated stents which are well known to those in the art. For example, a medicated stent containing the inhibitors useful in the methods of the present invention can be placed into the coronary vessel. The medicated stents slowly release the inhibitors over time. Local delivery by medicated stents is disclosed in Regar et al. Circulation 2002, 106:1949-1956, the method of which is hereby incorporated by reference.

Local delivery to the heart cardiac artery walls by balloon catheter is disclosed in Wallinsky and Thung Am. J. Coll. Cardiol. 15:475-481 (1990) and Azrin et al. Cathet. Cardiovasc. Diagn. 41:232-240 (1997), the methods of which are hereby incorporated by reference. This method is also useful for delivery of inhibitors according to the methods of the present invention.

In extreme emergencies, the inhibitors can be administered by injection directly into the heart.

Oral administration of inhibitors of the RAS for treating hypertension according to established regimens is not expected to be effective in myocardial ischemia or cardiac arrhythmia due to the need to block the RAS at the sympathetic nerve endings, where it is produced and where it acts in the heart. Oral dosages of RAS inhibitors used for antihypertensive treatments are not sufficiently bioavailable at these sites for effective treatment of myocardial ischemia or cardiac arrhythmia. Any orally administered RAS inhibitor would be quickly overwhelmed by the autocrine loop from the action of locally produced renin, ultimately generating Ang II, thus leading to norepinephrine release, which stimulates the Na⁺/H⁺ exchanger (NHE), further exacerbating the ischemia and/or arrhythmia and leading to yet more local renin production. The above described action of local RAS is in addition to the effect of circulating RAS, which normally leads to hypertension and cardiac muscle hypertrophy.

### Pharmaceutical Formulation

Any pharmaceutical formulation for local administration known in the art of pharmacy is suitable for administration of the inhibitors. The formulation may, for example, comprise conventional diluents, carriers, or excipients, etc., such as are known in the art. For example, the formulations may comprise one or more of the following: a stabilizer, a surfactant, preferably a nonionic surfactant, and optionally a salt and/or a buffering agent. The formulation may be delivered in the form of an aqueous solution.

The stabilizer may, for example, be an amino acid, such as for instance, glycine; or an oligosaccharide, such as for example, sucrose, tetralose, lactose or a dextran. Alternatively, the stabilizer may be a sugar alcohol, such as for instance, mannitol; or a combination thereof. Preferably the stabilizer or combination of stabilizers constitutes from about 0.1% to about 10% weight for weight of the inhibitor.

The surfactant is preferably a nonionic surfactant, such as a polysorbate. Some examples of suitable surfactants include Tween20, Tween 80, a polyethylene glycol or a polyoxyethylene polyoxypropylene glycol, such as Pluronic F-68 at from about 0.001% (w/v) to about 10% (w/v).

The salt or buffering agent may be any salt or buffering agent, such as for example, sodium chloride, or sodium/potassium phosphate, respectively. Preferably, the buffering agent maintains the pH of the pharmaceutical composition in the range of about 5.5 to about 7.5. The salt and/or buffering agent is also useful to maintain the osmolality at a level suitable for administration to a human or an animal. Preferably the salt or buffering agent is present at a roughly isotonic concentration of about 150 mM to about 300 mM.

The formulation may additionally contain one or more conventional additives. Some examples of such additives include a solubilizer such as, for example, glycerol; an ontioxidant such as for example, benzalkonium chloride (a mixture of quaternary ammonium compounds, known as “quats”), benzyl alcohol, chloretone or chlorobutanol; anaesthetic agent such as for example a morphine derivative; or an isotonic agent etc., such as described above. As a further precaution against oxidation or other spoilage, the pharmaceutical compositions may be stored under nitrogen gas in vials sealed with impermeable stoppers.

### General Methods for Isolating Renin

Renin can be isolated from cardiac synaptosomes by any method known to those in the art, such as isolation of proteins from solution or a gel. For example, renin can be isolated from solubilized fractions (e.g., atrial homogenates, synaptosomal preparations) by standard methods. Some suitable methods include precipitation and liquid/chromatographic protocols such as ion exchange, hydrophobic interaction and gel filtration. See for instance, Guide to Protein Purification, Deutscher, M. P. (Ed.) Methods Enzymol., 182, Academic Press, Inc., New York (1990) and Scopes, R. K. and Cantor, C. R. (Eds.), Protein Purification (3d), Springer-Verlag, New York (1994).

Alternatively, isolated renin can be obtained by separating the protein on preparative SDS-PAGE gels. The renin in the SDS gel can be identified using an antibody which recognizes circulating renin, such as those renin monoclonal antibodies available from Swant® Swiss Antibodies (Switzerland). Once the renin band is identified, the band is sliced from the gel and the protein is electroeluted from the polyacrylamide matrix by methods known in the art. The detergent SDS is removed from the protein by known methods, such as by dialysis or the use of a suitable column, such as the Extracti-Gel column from Pierce.

The activity of the isolated renin can be determined by any method known in the art. For example, renin is known to convert angiotensinogen to Ang I. Thus, activity of renin can be determined by, for instance, incubating isolated renin with angiotensinogen and determining if Ang I is produced.

### Antibody Production

Antibodies may be polyclonal or monoclonal, and may be produced by methods known in the art. For example, polyclonal antibodies can be isolated from mammals that have been inoculated with the protein or a functional analog in accordance with methods known in the art (Coligan, J. E, et al. (Eds.), Current Protocols in Immunology, Wiley Intersciences, New York, (1999)). Briefly, polyclonal antibodies may be produced by injecting a host mammal, such as a rabbit, mouse, rat, or goat, with the protein or peptide fragment. Sera from the mammal are extracted and screened to obtain polyclonal antibodies that are specific to the protein. In order to be useful, the peptide fragment must contain sufficient amino acid residues to define the epitope of the protein being detected.

If the peptide fragment is too short to be immunogenic, it may be conjugated to a carrier molecule. Some suitable carrier molecules include keyhole limpet hemocyanin and bovine serum albumen. Conjugation may be carried out by methods known in the art (Coligan, J. E. et al. (Eds.) Current Protocols in Immunology, Chapter 9, Wiley Intersciences, New York, (1999)). One such method is to combine a cysteine residue of the fragment with a cysteine residue on the carrier molecule.

The antibodies are preferably monoclonal. Methods for making monoclonal antibodies include the immunological method described by Kohler and Milstein in Nature 256:495-497 (1975) and by Campbell in “Monoclonal Antibody Technology, The Production and Characterization of Rodent and Human Hybridomas” in Burdon et al. (Eds.), Laboratory Techniques in Biochemistry and Molecular Biology, Volume 13, Elsevier Science Publishers, Amsterdam (1985); and Coligan, J. E, et al. (Eds.), Current Protocols in Immunology, Wiley Intersciences, New York, (1999); as well as the recombinant DNA method described by Huse et al., Science 246:1275-1281 (1989).

In order to produce monoclonal antibodies, a host mammal is inoculated with the protein as described above, and then boosted. Spleens are collected from inoculated mammals a few days after the final boost. Cell suspensions from the spleens are fused with a tumor cell in accordance with the general method described by Kohler and Milstein in Nature 256:495-497 (1975). See also Campbell, “Monoclonal Antibody Technology, The Production and Characterization of Rodent and Human Hybridomas” in Burdon et al. (Eds.), Laboratory Techniques in Biochemistry and Molecular Biology, Volume 13, Elsevier Science Publishers, Amsterdam (1985) and Coligan, J. E., et al. (Eds.), Current Protocols in Immunology, Wiley Intersciences, New York, (1999)).

### EXAMPLES

### Example 1

### Material and Methods

Source of human cardiac tissue. Specimens of right atrium (i.e., surgical waste tissue) were obtained from 34 patients undergoing cardiopulmonary bypass [29 males and 5 females, age 64.9±1.6 years; coronary artery bypass grafting (CABG) 30, valve replacement 4], following a protocol approved by the Institutional Review Board. Eight of the 30 CABG patients were chronically treated with β-adrenoceptor blocking agents. Preoperative treatment with β-blockers did not affect the ischemic release of NE. All patients chronically treated with ACE inhibitors were excluded from the study. At the time of surgery, a piece of atrial appendage measuring ˜1 cm³ was removed from the atriotomy site.

Incubation of atrial tissue. Atrial specimens were immediately transported to the laboratory in ice-cold oxygenated Krebs-Henseleit solution (KHS) of the following composition (mM): NaCl 118.2, KCl 4.83, CaCl₂ 2.5, MgSO₄ 2.37, KH₂PO₄ 1.0, NaHCO₃ 25, and glucose 11.1. After removal of fat and connective tissue, specimens were divided into several fragments (each weighing 24.5±1.1 mg, wet weight, measured at the end of incubation). Each fragment was incubated for 15 min at 37° C. in 2 ml of KHS gassed with 95% O₂ and 5% CO₂ (Po₂ ˜550 mm Hg, pH ˜7.4) containing the monoamine oxidase inhibitor pargyline (1 mM). Following the 15-min stabilization period, fragments were incubated for an additional 20 min in oxygenated KHS in the absence or presence of pharmacological agents.

Preparation of cardiac synaptosomes. Atrial specimens were freed from fat and connective tissue and minced in ice-cold 0.32 M sucrose containing 1 mM EGTA, pH 7.4, and 1 mM pargyline, to prevent enzymatic destruction of synaptosomal NE. Synaptosomes were isolated as previously described (Imamura et al., Circ. Res. 1995, 77:206-210). Briefly, minced tissue was digested with 120 mg collagenase (Type II, Worthington Biochemicals, Freehold, N.J.) per gm wet heart weight for 1 hr at 37° C. After low speed centrifugation (10 min at 120 g), the resulting pellet was suspended in 10 vol of 0.32 M sucrose and homogenized with a Teflon/glass homogenizer. The homogenate was spun at 650 g for 10 min and the pellet rehomogenized and respun. The pellet containing cellular debris was discarded, and the supernatants from the last two spins were combined and equally subdivided into 4 to 8 tubes and recentrifuged for 20 min at 20,000 g at 4° C. This pellet, which contained cardiac synaptosomes, was resuspended either in HEPES-buffered saline (HBS; 500 μl, normoxic conditions) or in glucose-free HBS which contained the reducing agent sodium dithionite (500 μl, ischemic conditions), and incubated in the absence or presence of angiotensinogen (for 1 hour) or other pharmacological agents for 20 min in a water bath at 37° C. prior to ischemia (see below). HBS contained 50 mM HEPES, pH 7.4, 144 mM NaCl, 5 mM KCl, 1.2 mM CaCl₂, 1.2 mM MgCl₂, 10 mM glucose. Each suspension functioned as an independent sample and was used only once. In every experiment, one sample was untreated (control, basal NE release).

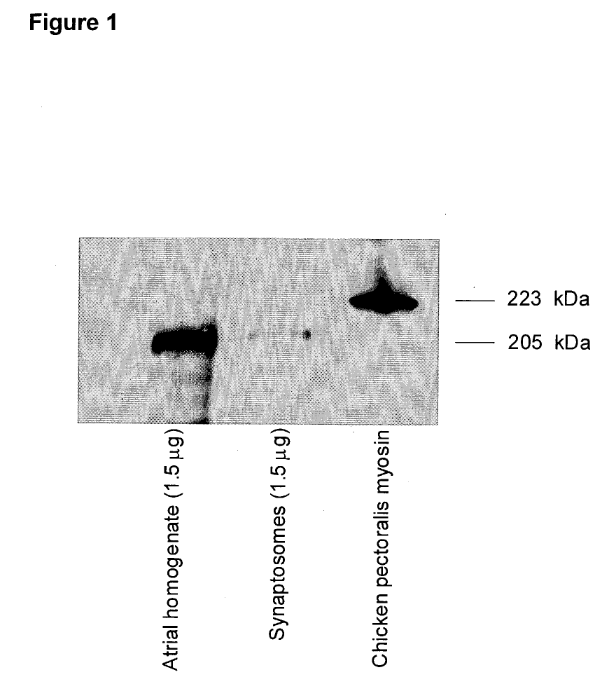

Purity of the synaptosomal preparation was verified by Western blot analysis of myosin using MF20, an antibody against sarcomeric myosin heavy chain (FIG. 1) (Bader et al., J. Cell. Biol. 1982, 95:763-770). Sarcomeric myosin was used as a marker of synaptosomal purity since it is present in myocardial tissue, but not in nerve endings. Equal amounts of protein (1.5 μg) for both atrial homogenate and synaptosomes were run in parallel and in triplicate on the same gel. The density of each band within a gel was analyzed using NIH Image (version 1.60). As shown in the immunoblot (see FIG. 1), detection of the 205 kDa band associated with myosin was barely detectable in the synaptosomal fraction as compared to the atrial homogenate (O.D.: 768±281 and 9,744±705 for synaptosomes and atrial homogenate, respectively; means±S.E.M.).

Induction of ischemia. Ischemia was induced by incubating either atrial fragments or synaptosomes for 70 min in glucose-free KHS (atrial tissue) or HBS (synaptosomes) bubbled with 95% N₂ and 5% CO₂, containing the reducing agent sodium dithionite (3 mM; PO₂ ˜0 mm Hg, pH ˜7.3; ischemic NE release) (Hatta et al. J. Pharmacol Exp. Ther. 1997, 283:494-500). Matched control fragments and synaptosomes were incubated for an equivalent length of time with oxygenated KHS and HBS, respectively (normoxic NE release). When drugs were used, they were continued throughout the entire normoxic and ischemic periods.

Norepinephrine assay. Incubating media were assayed for NE by high pressure liquid chromatography with electrochemical detection (Hatta et al. J. Pharmacol Exp. Ther. 1997, 283:494-500). Perchloric acid and EDTA were added to samples to achieve final concentrations of 0.01 N and 0.025%, respectively. The NE present in the effluent was adsorbed on acid-washed alumina adjusted at pH 8.6 with Tris-2% EDTA buffer, and then extracted into 150 μl of 0.1 N perchloric acid. These final sample aliquots were injected onto a 3 μm ODS reverse-phase column (3.2×100 mm, Bioanalytical System, West Lafayette, Ind.) with an applied potential of 0.65 V. The mobile phase consisted of monochloroacetic acid (75 mM), sodium EDTA (0.5 mM), sodium octylsulfate (0.5 mM) and acetonitrile (1.5%) at pH 3.0. The flow rate was 1.0 ml/min. No NE breakdown occurred during the 70-min ischemic period. Dihydroxybenzylamine was added to each sample as an internal standard prior to alumina extraction and used for calculation of the recovery during the extraction procedure. This recovery was 77% or better. The detection limit was approximately 0.2 pmol.

Western blotting. Human right atrial homogenate or synaptosomal preparations were mixed with 10 μl of 2× Novex Tris-glycine SDS sample buffer (Invitrogen, Carlsbad, Calif.) and boiled for 4-5 min. Samples were separated by electrophoresis on 4% and 10-20% gradient Tris-glycine SDS-polyacrylamide gels, for myosin and renin respectively. Electrophoresis was carried out at 50 V/gel for 60 min. Gels were soaked in transfer buffer (25 mM Tris-base, 0.2 M glycine and 20% methanol, pH 8.5) and electrotransferred onto 0.22 μm nitrocellulose membranes (Invitrogen) for 90 min at 200 V and 4° C. After transfer, the nitrocellulose was blocked for 1 hour in blocking buffer (Tris-buffered saline, TBS, containing 0.1% Tween 20, 5% w/v non-fat dry milk). Anti-sarcomeric myosin heavy chain antibody (MF20; Bader et al., 1982) or anti-renin antibody (BR1-5; Campbell et al., 1996) was incubated with the nitrocellulose overnight at 4° C., diluted 1:50,000 or 1:12,500 in primary antibody dilution buffer (TBS containing 0.1% Tween 20, 5% BSA), respectively. The nitrocellulose was washed three times with TBS, then horseradish peroxidase-coupled secondary antibody was added at a 1:2000 dilution in blocking buffer for 1 hour. After three further TBS washes, myosin or renin was detected using enhanced chemiluminescence (LumiGLO, Cell Signaling Inc., Beverly, Mass.). Chicken pectoralis myosin and mouse kidney extracts were used on appropriate gels as positive controls. Pre-stained molecular weight standards (Invitrogen) were included in all gels.

Statistics. Values are expressed as mean±S.E.M. Analysis by one-way ANOVA was used followed by Dunnett multicomparison testing. A value of P<0.05 was considered statistically significant.

Drugs and Chemicals. Human plasma angiotensinogen, desipramine hydrochloride (DMI), pepstatin-A and 5-(N-ethyl-N-isopropyl)-amiloride (EIPA) were purchased from Sigma-Aldrich Chemical Co (St. Louis, Mo.). Enalaprilat and EXP 3174 were from Merck Sharp & Dohme Research Laboratories (West Point, Pa.). BILA 2157BS was from Boehringer Ingelheim (Canada) Ltd., Research and Development (Laval, Qu [symbol] ., Canada). EXP 3174, DMI, pepstatin-A and EIPA were dissolved in dimethyl sulfoxide (DMSO). BILA 2157BS was dissolved in 0.02 M Na₂HPO₄ buffer. Further dilutions were made with distilled water; at the concentration used, DMSO and Na₂HPO₄ buffer did not affect NE release.

### Example 2

### Carrier-Mediated NE Release from Human Myocardium

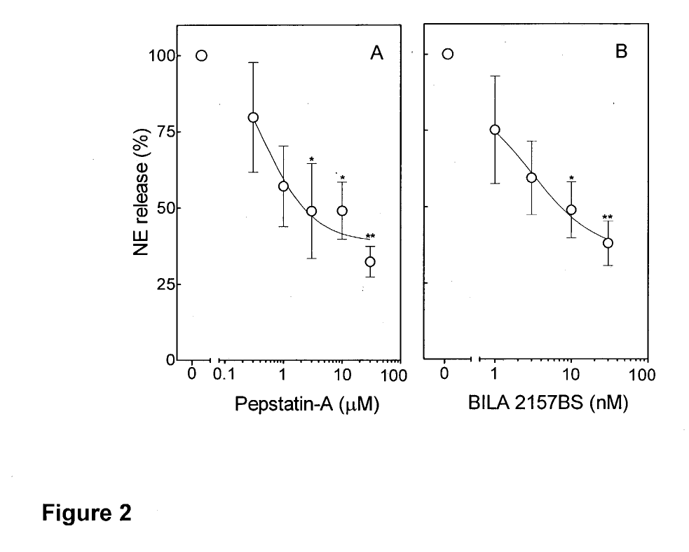

The incubation of human right atrial tissue for 70 min in glucose-free KHS in ischemic conditions (PO₂ ˜0 mmHg; pH ˜7.3), caused a seven-fold increase in the release of endogenous NE above basal level in normal oxygenated conditions (ischemic, 3.5±0.21 vs. basal, 0.5±0.11 pmol/mg protein; means±S.E.M.; n=12). As previously reported (Hatta et al. J. Pharmacol Exp. Ther. 1997, 283:494-500), this release is carrier-mediated, since it is Ca²⁺-independent and inhibited by the NE transporter inhibitor DMI. As shown in FIG. 2, inhibition of renin activity with either the aspartyl protease inhibitor pepstatin-A (panel A) or the more potent and selective renin inhibitor BILA 2157BS (panel B) caused a concentration-dependent decrease in NE release, which amounted to ˜70% with 30 μM pepstatin-A and ˜60% with 30 nM BILA 2157BS.

### Example 3

### Carrier-Mediated NE Release from Sympathetic Nerve Terminals Isolated from the Human Myocardium

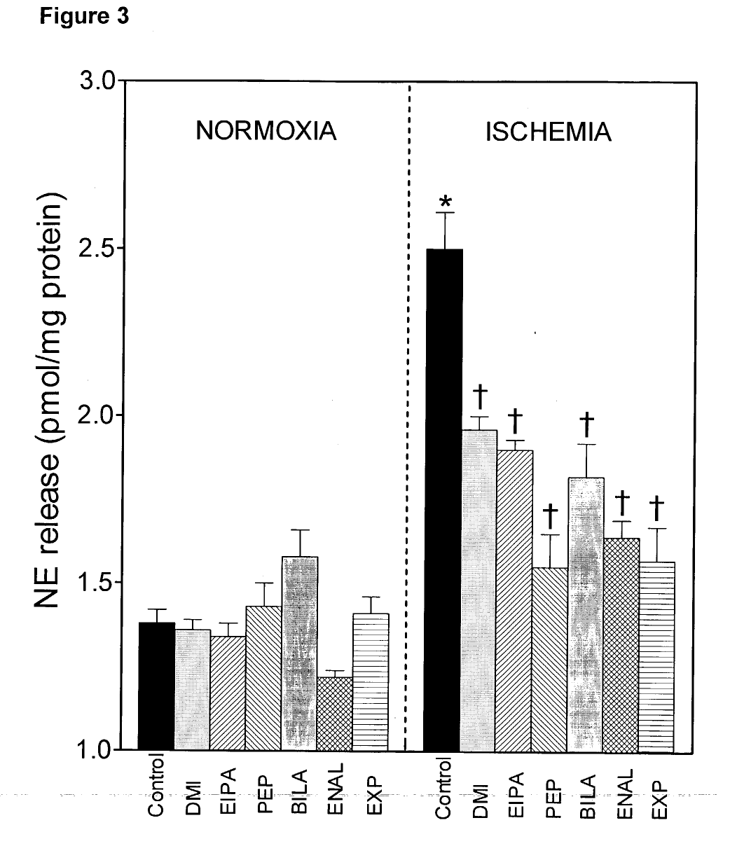

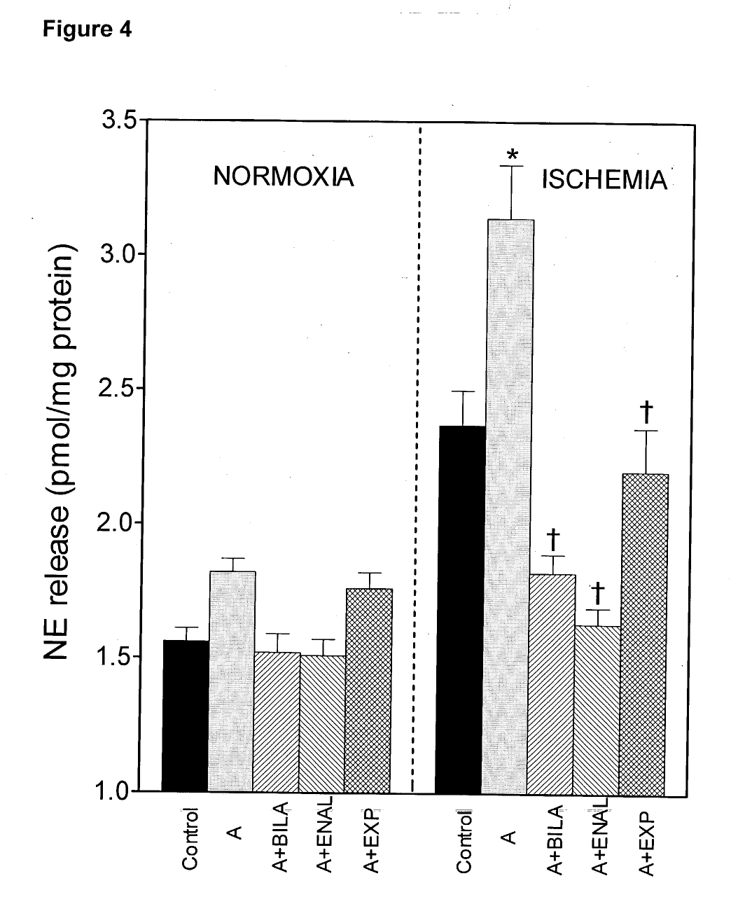

Sympathetic nerve endings (cardiac synaptosomes) were isolated from human right atrial tissue. As shown in FIGS. 3 and 4, incubation of human cardiac synaptosomes for 70 min in glucose-free KHS in ischemic conditions, elicited a significant release of endogenous NE (i.e., a ˜70% increase above basal level in normal oxygenated conditions). This release was inhibited by ˜50% by the NE transporter inhibitor DMI (300 nM) and by the inhibitor of the Na⁺/H⁺ exchanger EIPA (30 μM), indicating that it was carrier-mediated (FIG. 3). The increase in NE release caused by ischemia was markedly reduced (˜80%) by the ACE inhibitor enalaprilat (3 μM) and by the Ang II AT₁R antagonist EXP 3174 (300 nM), indicating the participation of endogenous Ang II in this process (FIG. 3). Furthermore, the renin inhibitors pepstatin-A (30 μM) and BILA 2157BS (100 nM) suppressed the enhancement in NE release elicited by ischemia by ˜70%, implying a role of renin in the neuronal formation of Ang II (FIG. 3).

A role of renin in the neuronal formation of Ang II was further supported by the findings depicted in FIG. 4. Incubation of human cardiac synaptosomes with angiotensinogen (400 nM) increased ischemic NE release by ˜70%. This increase was prevented by the renin inhibitor BILA 2157BS (100 nM), by the ACE inhibitor enalaprilat (3 μM) and by the Ang II AT₁R antagonist EXP 3174 (300 nM) (FIG. 4).

### Example 4

### Presence of Renin in Sympathetic Nerve Terminals Isolated from the Human Myocardium

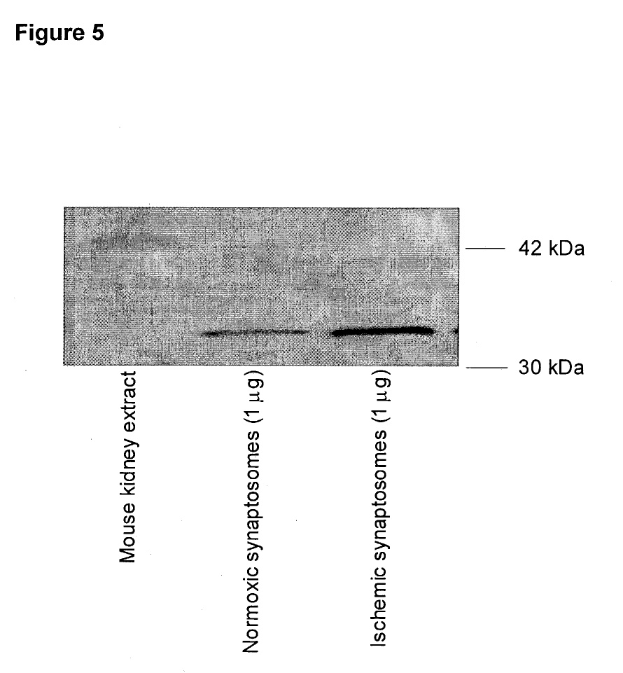

Sympathetic nerve terminals were screened by Western blot for the presence of renin with a specific antibody, BR1-5 (Campbell et al., Hypertension 1996, 27:1121-1133). Equal amounts of protein (1.0 μg) for both normoxic and ischemic synaptosomes were run in parallel and in triplicate on the same gel. FIG. 5 demonstrates that renin is present in sympathetic nerve endings isolated from the human heart and more importantly, that renin increases ˜three-fold with 70-min ischemia (O.D.: 820±135 and 2159±339 for normoxic and ischemic synaptosomes, respectively).

What is claimed is:

1. A method for treating a human suffering from myocardial ischemia, cardiac arrhythmia, or both, the method comprising administering locally to a heart of the human an effective amount of an enzyme inhibitor that inhibits formation of angiotensin II in the heart.

2. A method according to claim 1, wherein the enzyme inhibitor inhibits formation of angiotensin I.

3. A method according to claim 2, wherein the enzyme is renin.

4. A method according to claim 3, wherein the renin inhibitor is administered intrapericardially.

5. A method according to claim 3, wherein the renin inhibitor is pepstatin A, BILA 2157 BS, enalkiren 64662, CGP-38560A, ciprokiren Ro44-9375, A72517, A74273, remikiren Ro42-5892, Abbott-72517, Aliskiren, CI-992, EMD58265, or U 71038.

6. A method according to claim 5, wherein the renin inhibitor is a renin antibody.

7. A method according to claim 2, further comprising administering locally to the heart an effective amount of an inhibitor of receptor-mediated enhancement of neuronal NHE activity.

8. A method according to claim 7, wherein the inhibitor of receptor-mediated enhancement of neuronal NHE activity is an AT₁ receptor antagonist.

9. A method according to claim 8, wherein the AT₁ receptor antagonist is losartan, irbesartan, candesartan, cilexetil, valsartan, EXP3174 or an anti-AT₁ receptor antibody.

10. A method according to claim 2, further comprising administering locally to the heart an effective amount of an agonist of a receptor that mediates attenuation of neuronal NHE activity.

11. A method according to claim 10, wherein the receptor is a histamine H₃ receptor.

12. A method according to claim 10, wherein the receptor is an adenosine A₁ receptor.

13. A method according to claim 2, further comprising administering locally to the heart an effective amount of an inhibitor of NE release from the sympathetic nerve endings.

14. A method according to claim 13, wherein the inhibitor of NE release is bretylium.

15. A method according to claim 1, wherein the enzyme inhibitor inhibits formation of angiotensin II.

16. A method according to claim 1, wherein the enzyme is chymase.

17. A method according to claim 16, wherein the chymase inhibitor is administered intrapericardially.

18. A method according to claim 16, wherein the chymase inhibitor is α₁-anti-trypsin, chymostatin, BBI (Bowman-Birk inhibitor), Suc-Val-Pro-Phe(p)(OPh)(2), SQN-5, MNEI, NK3201, BCEAB (4-[1-[[bis-(4-methyl-phenyl)-methyl]-carbamoyl]-3-(2-ethoxybenzyl)-4-oxo-azetidine-2-yloyl]-benzoic acid, methyllinderone.

19. A method according to claim 16, wherein the chymase inhibitor is a chymase antibody.

20. A method according to claim 15, further comprising administering locally to the heart an effective amount of an inhibitor of receptor-mediated enhancement of neuronal NHE activity.

21. A method according to claim 20, wherein the inhibitor of receptor-mediated enhancement of neuronal NHE activity is an AT₁ receptor antagonist.

22. A method according to claim 21, wherein the AT₁ receptor antagonist is losartan, irbesartan, candesartan, cilexetil, valsartan, EXP3174 or an anti-AT₁ receptor antibody.

23. A method according to claim 15, further comprising administering locally to the heart an effective amount of an agonist of a receptor that mediates attenuation of neuronal NHE activity.

24. A method according to claim 23, wherein the receptor is a histamine H₃ receptor.

25. A method according to claim 23, wherein the receptor is an adenosine A₁ receptor.

26. A method according to claim 15, further comprising administering locally to the heart an effective amount of an inhibitor of NE release from the sympathetic nerve endings.

27. A method according to claim 26, wherein the inhibitor of NE release is bretylium.

28. A method according to claim 1, wherein the enzyme inhibitor is a combination of enzyme inhibitors which inhibit formation of Ang I and Ang II.

29. A method according to claim 28, wherein the enzyme is a combination of renin and chymase.

30. A method according to claim 29, wherein the renin inhibitor and the chymase inhibitor are administered intrapericardially.

31. A method according to claim 29, wherein the renin inhibitor is pepstatin A, BILA 2157 BS, enalkiren 64662, CGP-38560A, ciprokiren Ro44-9375, A72517, A74273, remikiren Ro42-5892, Abbott-72517, Aliskiren, CI-992, EMD58265, or U 71038.

32. A method according to claim 29, wherein the renin inhibitor is a renin antibody.

33. A method according to claim 29, wherein the chymase inhibitor is α₁-anti-trypsin, chymostatin, BBI (Bowman-Birk inhibitor), Suc-Val-Pro-Phe(p)(OPh)(2), SQN-5, MNEI, NK3201, BCEAB (4-[1-[[bis-(4-methyl-phenyl)-methyl]-carbamoyl]-3-(2-ethoxy-benzyl)-4-oxo-azetidine-2-yloyl]-benzoic acid, methyllinderone.

34. A method according to claim 29, wherein the chymase inhibitor is a chymase antibody.

35. A method according to claim 29, wherein the renin inhibitor is pepstatin A, BILA 2157 BS, enalkiren 64662, CGP-38560A. ciprokiren Ro44-9375, A72517, A74273, remikiren Ro42-5892, Abbott-72517, Aliskiren, CI-992, EMD58265, U 71038, or a renin antibody; and the chymase inhibitor is Suc-Val-Pro-Phe(p)(OPh)(2), SQN-5, MNEI, NK3201, BCEAB (4-[1-[[bis-(4-methyl-phenyl)-methyl]-carbamoyl]-3-(2-ethoxy-benzyl)-4-oxo-azetidine-2-yloyl]-benzoic acid, methyllinderone, or a chymase antibody.

36. A method according to claim 28, further comprising administering locally to the heart an effective amount of an inhibitor of receptor-mediated enhancement of neuronal NHE activity.

37. A method according to claim 36, wherein the inhibitor of receptor-mediated enhancement of neuronal NHE activity is an AT₁ receptor antagonist.

38. A method according to claim 37, wherein the AT₁ receptor antagonist is losartan, irbesartan, candesartan, cilexetil, valsartan, EXP3174 or an anti-AT₁ receptor antibody.

39. A method according to claim 28, further comprising administering locally to the heart an effective amount of an agonist of a receptor that mediates attenuation of neuronal NHE activity.

40. A method according to claim 39, wherein the receptor is a histamine H₃ receptor.

41. A method according to claim 39, wherein the receptor is an adenosine A₁ receptor.

42. A method according to claim 28, further comprising administering locally to the heart an effective amount of an inhibitor of NE release from the sympathetic nerve endings.

43. A method according to claim 42, wherein the inhibitor of NE release is bretylium.

44. An isolated human renin of about 32-36 kDa.

45. A isolated human renin according to claim 44, wherein the isolated renin is recognized by a renin antibody which binds circulating renin.

46. A isolated human renin according to claim 44, which is inhibited by pepstatin A, and BILA 2157 BS.

47. A isolated human renin according to claim 44, which is present in human heart.

48. A isolated human renin according to claim 47, which is present in human cardiac synaptosomes isolated from sympathetic nerve endings.

49. A isolated human renin according to claim 44, wherein the isolated renin is essentially pure.

50. A method for discovering drugs for treating myocardial ischemia or cardiac arrhythmia in humans, the method comprising:
providing a compound selected from a plurality of compounds for testing; and
determining whether the compound specifically inhibits renin.

51. A method according to claim 50, wherein, if the test compound specifically inhibits renin, the method further comprises testing whether the compound is also effective in treating myocardial ischemia or cardiac arrhythmia in humans.

52. A method for screening drug candidates for treating myocardial ischemia or cardiac arrhythmia in humans, the method comprising:
providing a compound from a plurality of compounds for testing; and;
determining whether the compound specifically inhibits chymase.

53. A method according to claim 52, wherein, if the test compound specifically inhibits chymase, the method further comprises testing whether the compound is also effective in treating myocardial ischemia or cardiac arrhythmia in humans.
